(19) United States
(12) Patent Application Publication
(10) Pub. No.: US 2017/0087036 A1
(43) Pub. Date: Mar. 30, 2017

(54) Systems and Methods of Preventing Bedsores

(71) Applicant: Martin Lynch, Rome, GA (US)

(72) Inventor: Martin Lynch, Rome, GA (US)

(21) Appl. No.: 14/868,806

(22) Filed: Sep. 29, 2015

(51) Int. Cl. A61F 15/00; A61B 17/58; A61F 5/30

(52) U.S. Cl. CPC A61F 15/008; A61F 5/30; A61B 17/58

(57) ABSTRACT

Example embodiments of the systems and methods of preventing bedsores take the pressure off of the soft tissue and direct it on the bone, which is made to handle the weight and the pressure. In example embodiments, to insert pins, incisions are made in the skin. A hole may then be drilled into the bone and a pin may be inserted into the hole in the bone. The pins may extend out through the skin. A support plate may be connected to the pins. In one embodiment, holes are drilled though the plate to accommodate the external fixation pins. In an alternative embodiment, one or more crossbars may be connected from pin to pin and then the plate may be clamped onto the one or more crossbars. In an example embodiment, a removable panel is located in the plate to provide access to tend to the wound.

### TECHNICAL FIELD

The present disclosure is generally related to medical devices and, more particularly, is related to a medical support apparatus.

### BACKGROUND

Bedsores, also called pressure sores or pressure ulcers, are injuries to skin and underlying tissue resulting from prolonged pressure on the skin. Bedsores most often develop on skin that covers bony areas of the body, such as the heels, ankles, hips and the tailbone.

People most at risk of bedsores are those with a medical condition that limits their ability to change positions, requires them to use a wheelchair or confines them to a bed for a long time. Bedsores can develop quickly and are often difficult to treat.

For people who are confined to a bed, common sites include the back or sides of the head; rim of the ears; shoulders or shoulder blades; hip, lower back, or tailbone; and heels, ankles, and skin behind the knees.

Bedsores are caused by pressure against the skin that limits blood flow to the skin and nearby tissues. Other factors related to limited mobility can make the skin vulnerable to damage and contribute to the development of pressure sores. Three primary contributing factors are sustained pressure, friction, and sheer.

When the skin and the underlying tissues are trapped between bone and a surface such as a wheelchair or a bed, the sustained pressure may be greater than the pressure of the blood flowing in the tiny vessels (capillaries) that deliver oxygen and other nutrients to tissues. Without these essential nutrients, skin cells and tissues are damaged and may eventually die. This kind of sustained pressure tends to happen in areas that aren't well padded with muscle or fat and that lie over a bone, such as the spine, tailbone, shoulder blades, hips, heels, and elbows.

Friction is the resistance to motion. Friction may occur when the skin is dragged across a surface, such as when a patient changes position or a care provider moves the patient. The friction may be even greater if the skin is moist. Friction may make fragile skin more vulnerable to injury.

Shear occurs when two surfaces move in the opposite direction. For example, when a hospital bed is elevated at the head, the patient may slide down in bed. As the tailbone moves down, the skin over the bone may stay in place—essentially pulling in the opposite direction. This motion may injure tissue and blood vessels, making the site more vulnerable to damage from sustained pressure.

People may be at risk of developing pressure sores if they have difficulty moving and are unable to easily change position while seated or in bed. Previous solutions to dealing with bedsores include repositioning of the patient and use of special beds. There are heretofore unaddressed needs with these previous solutions.

### SUMMARY

Example embodiments of the present disclosure provide systems of preventing bedsores. Briefly described, in architecture, one example embodiment of the system, among others, can be implemented as follows: a plurality of external fixation pins configured to: attach to a weight bearing bone; and extend through the skin; and at least one support structure configured to attach to at least one of the plurality of external fixation pins.

Embodiments of the present disclosure can also be viewed as providing methods for preventing bedsores. In this regard, one embodiment of such a method, among others, can be broadly summarized by the following steps: attaching a plurality of external fixation pins to a weight bearing bone , the external fixation pins extending through the skin; and attaching at least one support structure to at least one of the plurality of external fixation pins.

### BRIEF DESCRIPTION OF THE DRAWINGS

FIG. 1 provides a perspective view of an example embodiment of a system of preventing bedsores.

FIG. 2 provides a perspective view of an example embodiment of a system of preventing bedsores.

FIG. 3 provides a perspective view of an example embodiment of the system of preventing bedsores of FIG. 2.

FIG. 4 provides a perspective view of an example embodiment of a system of preventing bedsores.

FIG. 5 provides a perspective view of an example embodiment of a system of preventing bedsores.

FIG. 6 provides a perspective view of an example embodiment of a system of preventing bedsores.

FIG. 7 provides a flow diagram of an example embodiment of a method of preventing bedsores.

### DETAILED DESCRIPTION

Embodiments of the present disclosure will be described more fully hereinafter with reference to the accompanying drawings in which like numerals represent like elements throughout the several figures, and in which example embodiments are shown. Embodiments of the claims may, however, be embodied in many different forms and should not be construed as limited to the embodiments set forth herein. The examples set forth herein are non-limiting examples and are merely examples among other possible examples.

On of the most common locations for bedsores is tailbone area or around the spine. There is a lot of turning and shifting of weight that should occur every couple of hours or so to prevent the bedsores, but there is still a propensity for the bedsores to still develop while in nursing homes.

Bedsores fall into one of four stages based on their severity. The National Pressure Ulcer Advisory Panel, a professional organization that promotes the prevention and treatment of pressure ulcers, defines each stage as follows. In Stage I, the beginning stage of a pressure sore. the following characteristics are presented. The skin is not broken. The skin may appear red on people with lighter skin color, and the skin does not briefly lighten (blanch) when touched. On people with darker skin, the skin may show discoloration, and the sore does not blanch when touched. The site may be tender, painful, firm, soft, warm or cool compared with the surrounding skin.

In Stage II, the outer layer of skin (epidermis) and part of the underlying layer of skin (dermis) may be damaged or lost. The wound may be shallow and pinkish or red. The wound may look like a fluid-filled blister or a ruptured blister.

In Stage III, the ulcer is a deep wound. The loss of skin usually exposes some fat. The ulcer looks crater-like. The bottom of the wound may have some yellowish dead tissue. The damage may extend beyond the primary wound below layers of healthy skin.

In Stage IV, an ulcer shows large-scale loss of tissue. The wound may expose muscle, bone or tendons. The bottom of the wound likely contains dead tissue that's yellowish or dark and crusty. The damage often extends beyond the primary wound below layers of healthy skin. A pressure ulcer is considered unstageable if its surface is covered with yellow, brown, black or dead tissue such that it is not possible to determine the depth of the wound.

Once a bedsore reaches stage III or IV, current treatments may include a fluidized bed in which air is blown through that makes the bed similar to a waterbed. These beds may be very costly and are usually rented on a daily basis. Example embodiments of the systems and methods of preventing bedsores include the use of external fixation pins, or modified pins, widely used in orthopedic surgeries. Various additions may be made to connect crossbars for frameworks using these pins as support. The external fixation pins are usually used for broken bones and fractures to hold the bone fragments into position while the bone heals. With the example embodiments of preventing bedsores disclosed herein, the external fixation pins may be inserted into the normal weight bearing prominence of the pelvis so that the weight is born by the bones that normally support the weight. However, a frame may be constructed using the external fixation pins so that the weight is born by the bone not the soft tissues.

Example embodiments of the systems and methods of preventing bedsores take the pressure off of the soft tissue and direct it on the bone, which is made to handle the weight and the pressure. In example embodiments, to insert the pins, incisions are made in the skin. A hole may then be drilled into the bone and a pin may be inserted into the hole in the bone. The pins may be loaded into a drill and then drilled into the bone. The pins may extend out through the skin. In situ infections may occur but are fairly easy to treat. If the pin is removed, infection treatment is straight-forward. A support plate may be connected to the pins. The plate may be constructed of hard, durable plastic. An example embodiment uses a thick plastic that may be shaped in a standard shape or it may be fit to a particular patient. In one embodiment, holes are drilled though the support plate to accommodate the external fixation pins. In an alternative embodiment, one or more crossbars may be connected from pin to pin and then the plate may be clamped onto the one or more crossbars. In an example embodiment, a removable panel is located in the plate to provide access to tend to the wound.

Example embodiments of the external fixation pins include various lengths and various materials. Various means of connecting the support structure include non-limiting examples of a threaded nut, clamp, ball and socket, and a cotter pin. In an example embodiment, a crossbar is attached to the pins and the support structure is attached to the crossbar, for example, with a clamping mechanism. In an alternative embodiment, a custom plate may be designed using a computer generated model that fits on the pins. The pins may extend through the plate and the plate is affixed to the pins. Again, various means of affixing the custom plate include non-limiting examples of a threaded nut, clamp, ball and socket, and a cotter pin. In an example embodiment standard size plates may accommodate various sized people.

In an alternative embodiment, the pins include a mushroom-type cap on the end. The mushroom-type ends act as the support to keep the pressure off of the localized area and obviate the use of the support plate. Although the pelvis bone is used in most of the drawings and the description for illustrative purposes, example embodiments of the systems and methods of preventing bedsores disclosed herein may be used for any area of the body. The purpose of the example embodiments disclosed herein is to have the bone bear the weight so that the soft tissue is not bearing the pressure of the weight of the body between the boney prominences and a bed or other structure. The support structure may be designed to distribute the weight.

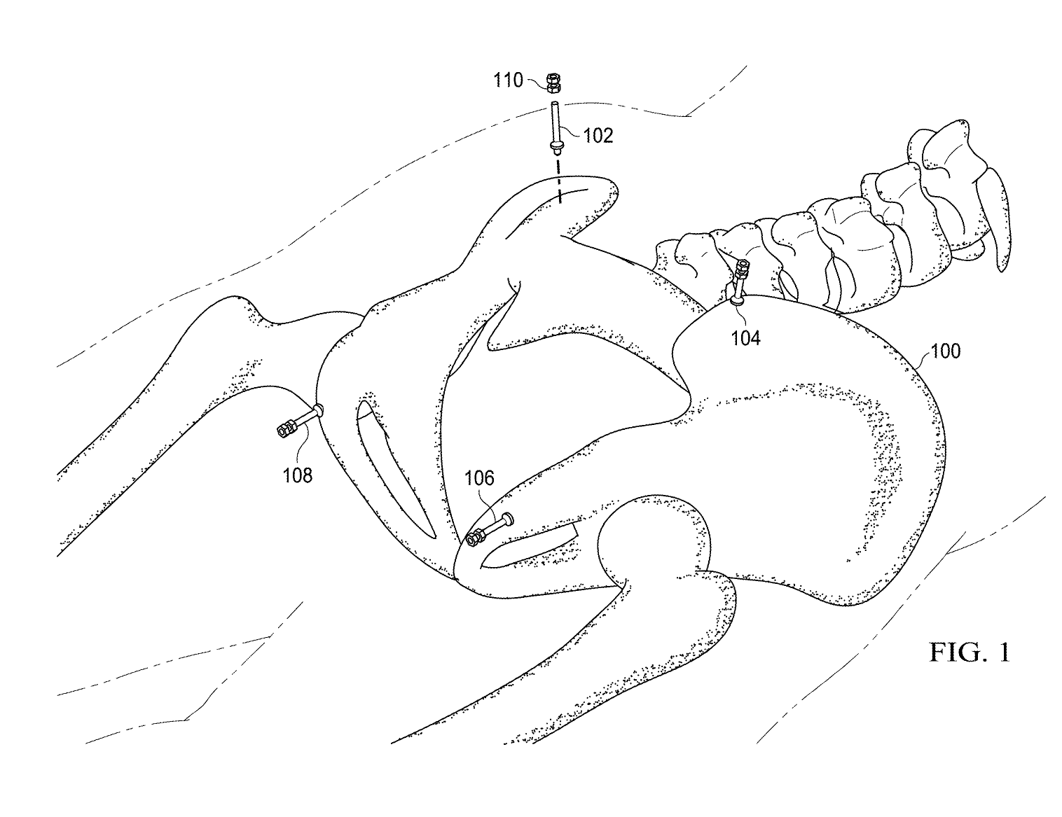

FIG. 1 provides a perspective view of an example embodiment of a system of preventing bedsores, including bone 100, pins 102, 104, 106, 108, and attachment means 110. In example embodiments, to insert pins 102, 104, 106, and 108, incisions are made in the skin. Holes may then be drilled into bone 100, the pelvic bone, for example, and pins 102, 104, 106, and 108 may be inserted into the holes in bone 100. Pins 102, 104, 106, and 108 may be loaded into a drill and then drilled into bone 100. The pins may extend out through the skin. Although bone 100 may be generally considered to be the pelvic bone, pins 102, 104, 106, and 108 (and additional pins) may be inserted into other bones as well. Attachment means 110 may be used to attach the support structure to pin 102 (with similar means for pins 104, 106, and 108).

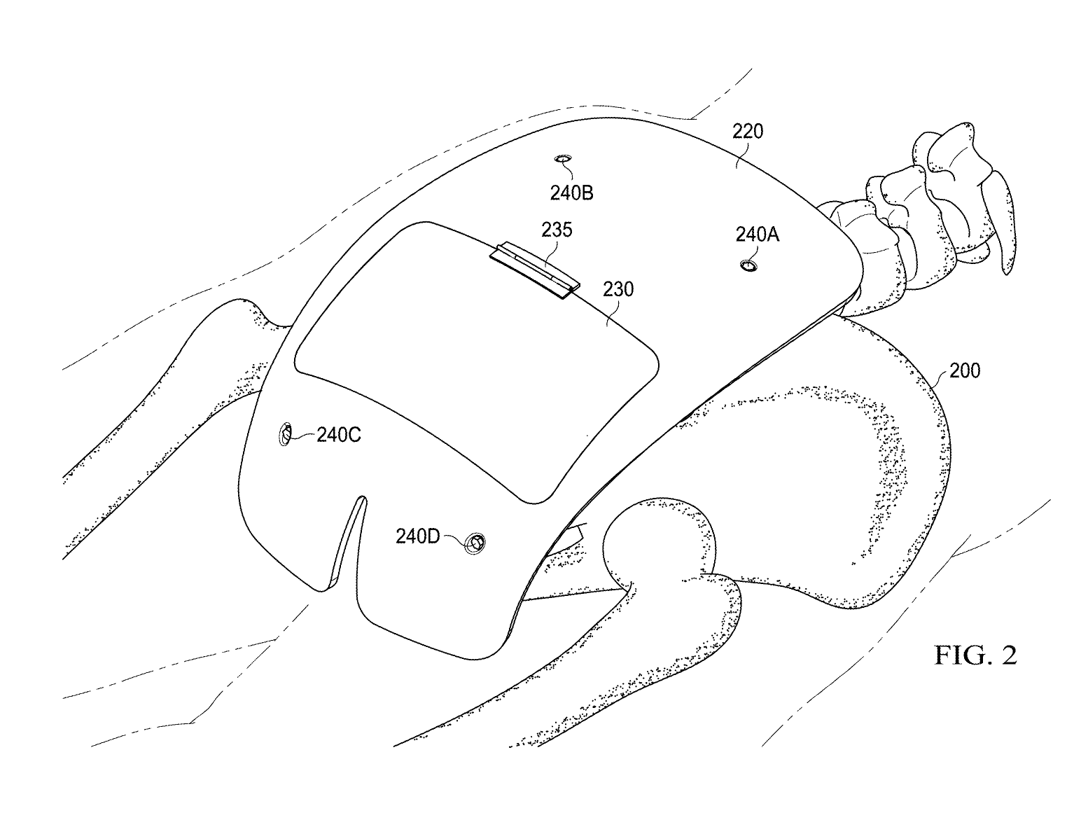

FIG. 2 provides a perspective view of an example embodiment of a system of preventing bedsores, including bone 200, support plate 220, access panel 230, hinge 235, and attachment locations 240A, 240B, 240C, and 240D. Support plate 220 fits onto the pins that are connected to bone 200. In an example embodiment support plate 220 connects to the pins at attachment locations 240A, 240B, 240C, and 240D. Access panel 230 is placed in support plate 220 and attached to support plate 220 with hinge 235. In an example embodiment, with activation of hinge 235, access plate 230 is allowed to swing open, providing access to the skin and to the pins underneath support plate 220. In an alternative embodiment, hinge 235 is not used, and access plate 230 comprises a pop-out cover. The attachment of support plate 220 to the pins inserted into bone 220 may leave space between support plate 220 and the skin of the patient. This relieves the skin of bearing the force of the weight, thereby preventing/reducing the occurrences of bedsores.

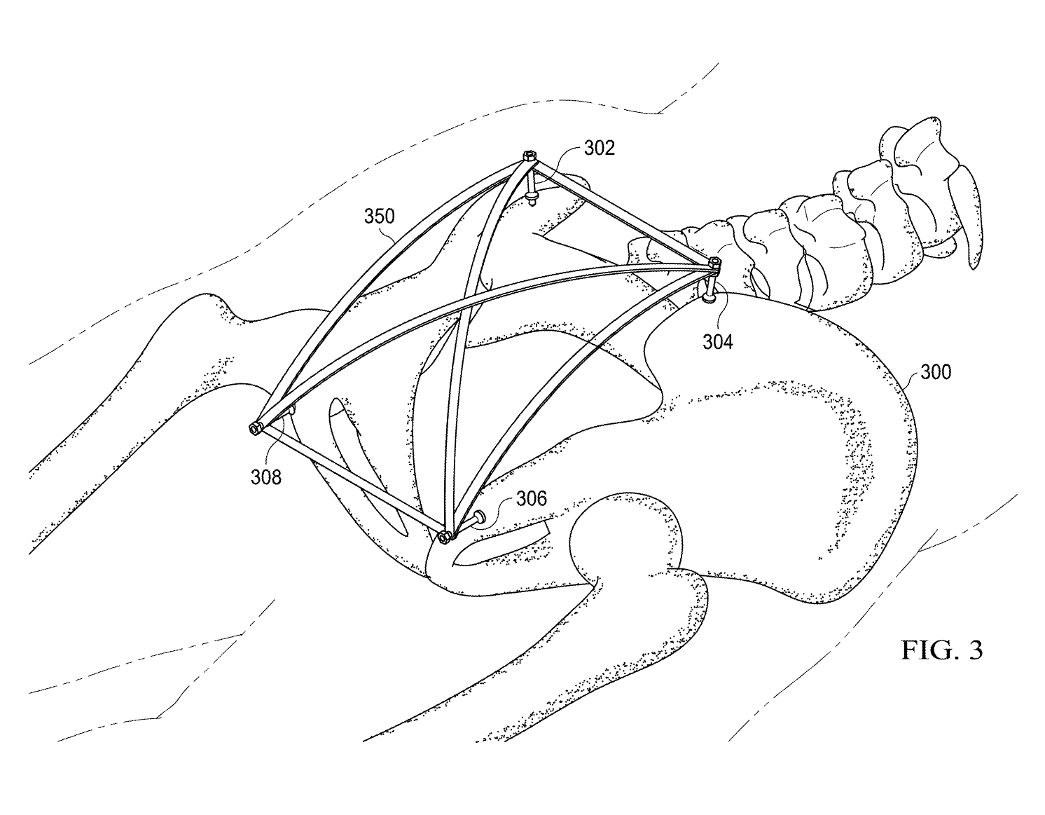

FIG. 3 provides a perspective view of an example embodiment of a system of preventing bedsores, including bone 300, pins 302, 304, 306, 308, and support frame 350. Support frame 350 connects to pins 302, 304, 306, 308 that are connected to bone 300. In an example embodiment, support frame 350 provides support to relieve the skin of the patient from bearing the force of the weight, thereby preventing/reducing the occurrences of bedsores. Open support frame 350 also allows additional airflow and better access to the skin and pins 302, 304, 306, 308.

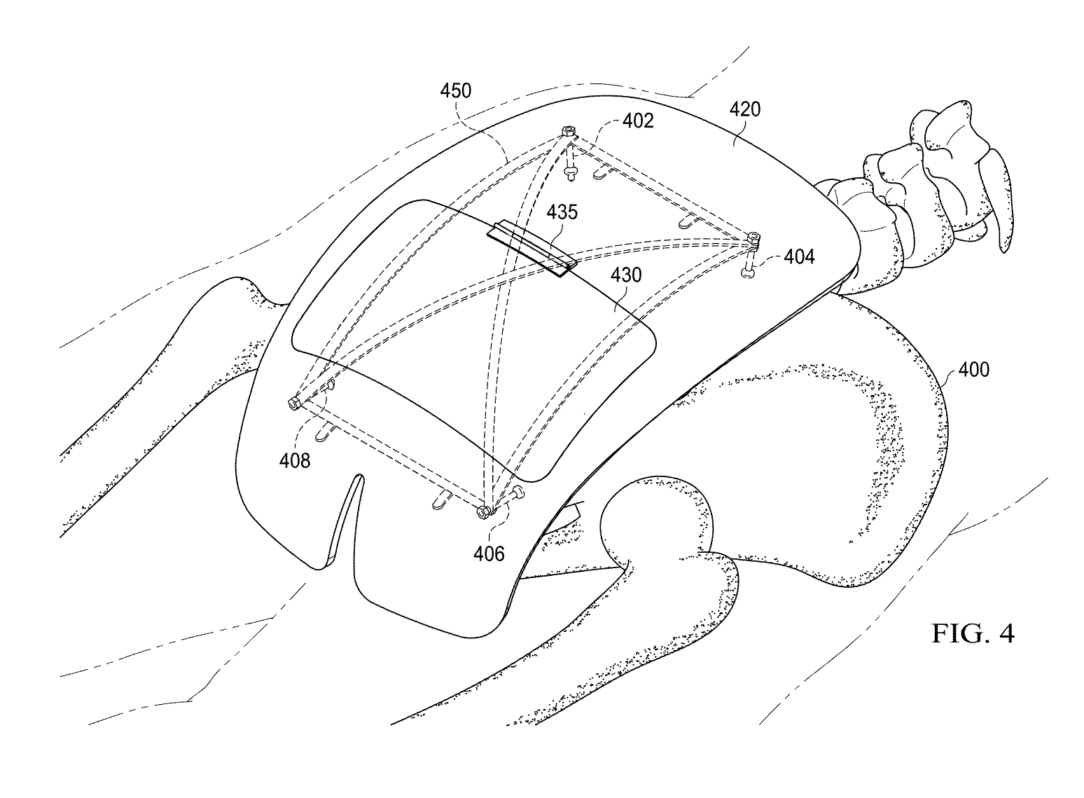

FIG. 4 provides a perspective view of an example embodiment of a system of preventing bedsores, including bone 400, pins 402, 404, 406, 408, support plate 420, support frame 450, access plate 430, and hinge 435. Support frame 450 connects to pins 402, 404, 406, 408 that are connected to bone 400. In an example embodiment, support frame 420 attaches to support frame 450, by any known attachment means. Access plate 430 is attached to support plate 420 with hinge 435 or other attachment means. Access plate 430 may swing out along hinge 435, thereby providing access to the skin and pin insertion points underneath support plate 420. In an alternative embodiment, hinge 435 is not used and access plate 430 comprises a pop-out plate. In an example embodiment, support plate 420 provides support to relieve the skin of the patient from bearing the force of the weight, thereby preventing/reducing the occurrences of bedsores. The attachment of support plate 420 to pins 402, 404, 406, 408 inserted into bone 420 may leave space between support plate 420 and the skin of the patient.

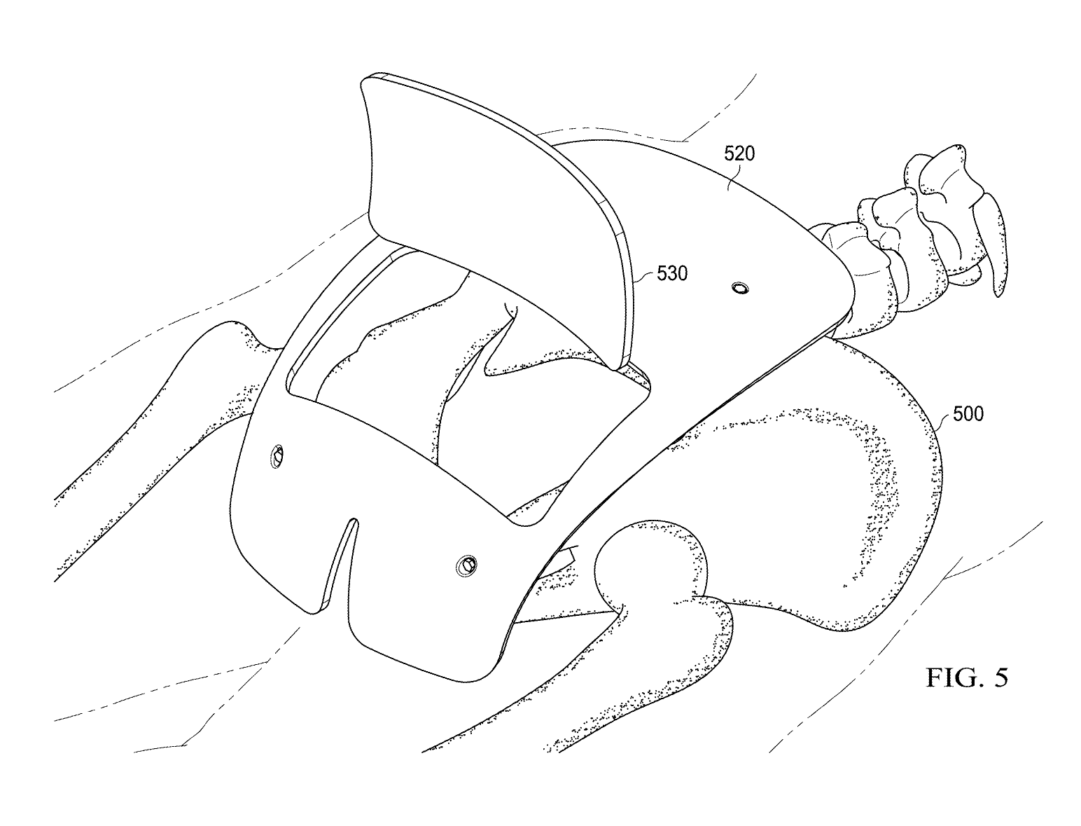

FIG. 5 provides a perspective view of an example embodiment of a system of preventing bedsores, including bone 500, support plate 520 and access plate 530. Access plate 530 is provided in an open position, allowing access to the skin and pins underneath support plate 520.

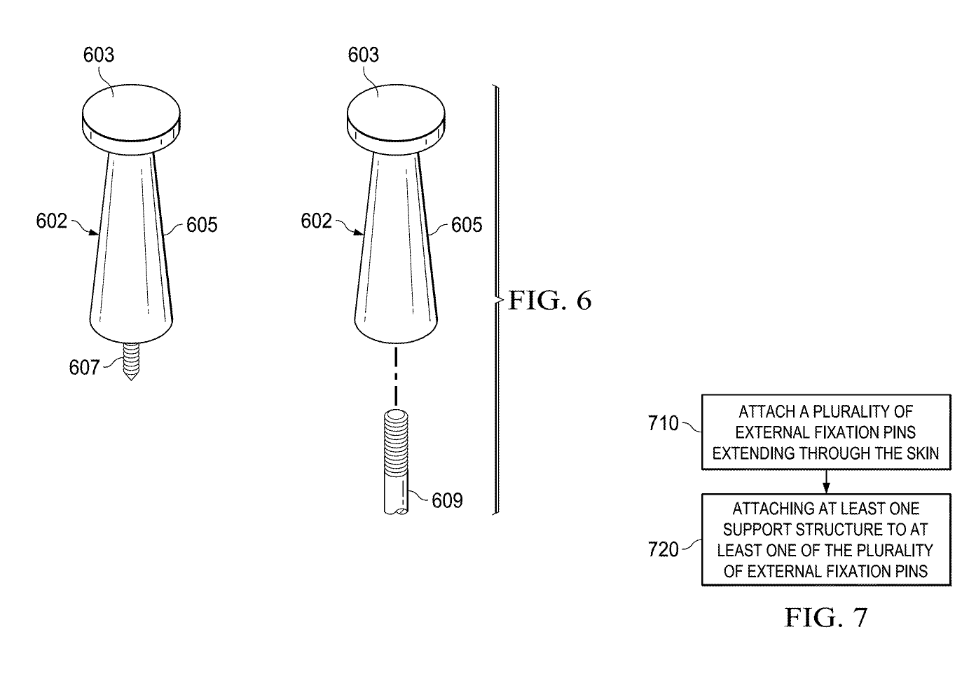

FIG. 6 provides a perspective view of an alternative embodiment of a system of preventing bedsores, including pin 609 with support stud 602. Support stud 602 may include shaft 605 and support surface 603. In an example embodiment, shaft 605 connects to pin 609, for example, by threads on pin 609. In an alternative embodiment, pin 602 has its own threaded pin 607 that screws directly into the bone. In an example embodiment, support surface 603 has a mushroom-like shape and bears the weight directly, providing space between the skin and, for example, a bed. In an example embodiment, one or more support studs 602 may be used as a stand alone method to reduce pressure by virtue of support surface 603 of support stud 602 and are not attached to a support frame or support plate. Support studs 602 may be used in pressure points other than the sacrum such as an ischial pressure ulcer from prolonged sitting.

FIG. 7 provides a flow diagram of an example embodiment of a method of preventing bedsores. In block 710, a plurality of external fixation pins are attached to a weight bearing bone, the external fixation pins extending through the skin. In block 720, at least one support structure is attached to at least one of the plurality of external fixation pins.

The flow chart of FIG. 7 shows the operation of a possible implementation of the method of preventing bedsores. It should also be noted that in some alternative implementations, the functions noted in the blocks may occur out of the order noted in FIG. 7. For example, two blocks shown in succession in FIG. 7 may in fact be executed substantially concurrently or the blocks may sometimes be executed in the reverse order, depending upon the functionality involved.

Although the present disclosure has been described in detail, it should be understood that various changes, substitutions and alterations can be made thereto without departing from the spirit and scope of the disclosure as defined by the appended claims.

Therefore, at least the following is claimed:

1. A system comprising:
a plurality of external fixation pins configured to:
attach to a weight bearing bone; and
extend through the skin; and
at least one support structure configured to attach to at least one of the plurality of external fixation pins.

2. The system of claim 1, wherein the support structure attaches to each of the plurality of external fixation pins.

3. The system of claim 1, wherein the support structure comprises an access port allowing for access to the insertion areas.

4. The system of claim 3, wherein the access port comprises at least one of a pop-out cover and a hinged cover.

5. The system of claim 1, wherein the external fixation pins fit through holes in the support structure.

6. The system of claim 1, wherein the support structure comprises a frame attached to the plurality of external fixation pins.

7. The system of claim 6, further comprising a support plate configured to attach to the frame.

8. The system of claim 1, wherein each of the at least one support structure attaches to exactly one of the plurality of external fixation pins.

9. The system of claim 8, wherein the support structures comprise at least one attachment mechanism of fitting over the external fixation pins or screwing into the external fixation pins.

10. A method comprising:
attaching a plurality of external fixation pins to a weight bearing bone , the external fixation pins extending through the skin; and
attaching at least one support structure to at least one of the plurality of external fixation pins.

11. The method of claim 10, wherein the attaching comprises attaching the support structure to each of the external fixation pins.

12. The method of claim 10, further comprising providing an access port in the support structure.

13. The method of claim 12, wherein the access port comprises at least one of a pop-out cover and a hinged cover.

14. The method of claim 10, wherein attaching the support structure comprises fitting the support structure onto the external fixation pins such that the external fixation pins fit through holes in the support structure.

15. The method of claim 10, wherein attaching comprises attaching a frame to the plurality of external fixation pins.

16. The method of claim 15, further comprising attaching a support plate to the frame.

17. The method of claim 10, wherein each of the at least one support structures attaches to exactly one of the plurality of external fixation pins.

18. The method of claim 17, wherein the support structures comprise at least one attachment mechanism of fitting over the external fixation pins and screwing into the external fixation pins.

19. A system comprising:
a plurality of external fixation pins configured to:
attach to a weight bearing bone; and
extend through the skin; and
at least one support structure configured to:
attach to at least one of the plurality of external fixation pins; and
fit a shape and size of a body being attached to.

20. The system of claim 19, wherein the support structure comprises a frame attached to the plurality of external fixation pins and a support plate configured to attach to the frame.
